(19) United States
(12) Patent Application Publication
(10) Pub. No.: US 2017/0157366 A1
(43) Pub. Date: Jun. 8, 2017

(54) URINARY CATHETERS, SYSTEMS AND METHODS FOR USE DURING TREATMENT OF THE PROSTATE

(71) Applicants: Benny ASSIF, Ramat HaSharon (IL); Alexander Silberman, Tirat Carmel (IL)

(72) Inventors: Benny ASSIF, Ramat HaSharon (IL); Alexander Silberman, Tirat Carmel (IL)

(21) Appl. No.: 14/957,959

(22) Filed: Dec. 3, 2015

(51) Int. Cl. A61M 25/10; A61M 25/00

(52) U.S. Cl. CPC A61M 25/10; A61M 25/0017; A61M 25/0043; A61M 2205/058; A61M 2205/3368; A61M 2025/006

(57) ABSTRACT

A urinary catheter is configured for avoiding heat buildup in, or even cooling, surrounding tissues when the prostate is subjected to treatment radiation such as ultrasound. The catheter includes a region transparent to ultrasound, and through which a cooling fluid (e.g., chilled water) may, in some embodiments, be recirculated during treatment. This region spans the prostate when the catheter is fully inserted (with the tip residing in the patient's bladder and a terminal balloon inflated to avoid retraction of the catheter).

### FIELD OF THE INVENTION

The present invention relates, generally, to ultrasound treatment of the prostate gland and, more specifically, to systems and methods for regulating temperature during focused ultrasound therapy.

### BACKGROUND

Certain types of body tissues, such as tumors, can be destroyed by heat. One way to apply thermal energy to internal body tissue is to focus high-intensity ultrasound waves into the tissue using, e.g., a phased array of piezoelectric transducer elements. Such treatment can ablate the tissue and thereby reduce or even eliminate the need for invasive surgery to remove it. For effective treatment, it is important that a sufficient thermal dose be reached during each ultrasound application to ablate or otherwise destroy the portion of the target tissue being heated. At the same time, it is important to regulate the temperature to avoid painful or damaging heat build-up in healthy tissues surrounding the target tissue, or even excessive heat in the target tissue. For example, ultrasound treatment of the prostate gland can involve eradicating a malignant lesion or selectively removing tissue to treat benign prostatic hyperplasia (BPH). In either case, excessive heat can injure the patient by destroying healthy tissue, such as the urethra and/or the urethral sphincter.

During prostate treatment, a urinary catheter such as a Foley device is typically passed through the patient's urethra and into his bladder. The Foley catheter has two separate channels, or lumens, one of which allows urine collected through a distal port to drain out into a collection bag. The other lumen permits inflation of a balloon, also located at the distal end, to fix the catheter position within the body (e.g., retaining the end of the catheter within the bladder). Foley catheters are commonly made from silicone or natural rubber (latex) with wall thicknesses of 2-3 mm. They are very flexible in the transverse direction and very strong in the longitudinal direction, i.e., no guide wire is required to introduce or to extract the catheter. Unfortunately, they also absorb about 95% of ultrasonic radiation incident thereon and, as a result, are strongly heated by the treatment beam. That heat may be sufficient to destroy urethral tissue and surrounding prostate tissue (which typically is not the tissue to be ablated). As a result, the risk of injury can preclude the use of ultrasound as a treatment modality altogether if catheterization is required.

Accordingly, there is a need for urinary catheters that avoid the risk of catheter-induced patient injury during prostate-treatment procedures.

### SUMMARY

The present invention provides, in various embodiments, a urinary catheter configured to permit large ultrasound power levels to be applied to the prostate without harm to surrounding tissues. In various embodiments, the catheter includes a region that, with the catheter in place, spans the region through which ultrasound is applied and is substantially (e.g., at least 90%, or 95%, or in some cases 99%) transparent to ultrasound energy. In some embodiments only this portion of the catheter is transparent to ultrasound and the rest of the catheter is constructed of more traditional materials, while in other embodiments, substantially the entire catheter is made of the material transparent to ultrasound.

The catheter may also be configured to permit a cooling fluid (e.g., chilled water) to be recirculated through the ultrasound-spanning region during treatment. In general, this region of the catheter spans the prostate when the catheter is fully inserted (with the tip residing in the patient's bladder and a terminal balloon inflated to avoid retraction of the catheter).

Accordingly, in a first aspect, the invention pertains to catheter configured for evacuating urine from a patient during a prostate procedure. In various embodiments, the catheter comprises an elongated tube for insertion through the patient's urinary tract. The tube has an outer wall and a urine-evacuation lumen therethrough and comprises a plurality of adjacent regions through which the urine-evacuation lumen passes. In particular, a distal region is configured for insertion into the patient's bladder and includes a terminal urine entry port in fluid communication with the urine-evacuation lumen and an inflatable balloon for retaining the port within the bladder. At least a prostate-spanning region, proximally adjacent to the distal region and having a length sized to span the patient's prostate gland with the balloon inflated and the urine entry port within the patient's bladder, is substantially transparent to ultrasound.

A cooling region, coextensive with the prostate-spanning region, comprises a fluid chamber fluidically isolated from and surrounding the urine-evacuation lumen and in thermal contact with the outer wall of the catheter. A proximal region is proximally adjacent to the cooling region, and includes (i) within the outer wall of the catheter, an inlet conduit in fluid communication with the fluid chamber for conducting a fluid thereto and and outlet conduit in fluid communication with the fluid chamber for conducting a fluid therefrom, and (ii) interfaces for fluidly coupling the urine-evacuation lumen to a collection container and the inlet and outlet conduits to a cooling and recirculation pump.

In various embodiments, the outer wall in the prostate-spanning region comprises or consists essentially of a hard polymeric material, e.g, MYLAR or a rigid polyurethane. The fluid chamber may be coaxial with the urine-evacuation channel. For example, the fluid chamber may have an annular cross-section and is bounded by an outer surface of the urine-evacuation channel and an inner surface of the outer wall. Alternatively, the fluid chamber may have a pair of concentric channels separated by a porous membrane, where an outer channel of the fluid chamber is interfaced to the inlet conduit and an inner channel of the fluid chamber is interfaced to the outlet conduit. In other embodiments, the fluid chamber comprises a plurality of tortuously arranged channels.

In another aspect, the invention relates to a system for evacuating urine from a patient during a prostate procedure and cooling the prostate. In various embodiments, the system comprises a catheter comprising an elongated tube for insertion through the patient's urinary tract. The tube has an outer wall and a urine-evacuation lumen therethrough and comprises a plurality of adjacent regions through which the urine-evacuation lumen passes. A distal region is configured for insertion into the patient's bladder and includes a terminal urine entry port in fluid communication with the urine-evacuation lumen and an inflatable balloon for retaining the port within the bladder; a cooling region is proximally adjacent to the distal region and has a length sized to span the patient's prostate gland with the balloon inflated and the urine entry port within the patient's bladder; the cooling region comprises a fluid chamber fluidically isolated from and surrounding the urine-evacuation lumen and in thermal contact with the outer wall of the catheter. A proximal region is proximally adjacent to the cooling region and includes, within the outer wall of the catheter, an inlet conduit in fluid communication with the fluid chamber for conducting a fluid thereto and and outlet conduit in fluid communication with the fluid chamber for conducting a fluid therefrom. The system further includes a recirculation assembly configured to circulate a cooling fluid through the fluid chamber of the catheter without disturbing a flow of urine through the urine-evacuation lumen. In various embodiments, the recirculation assembly comprises a cooling unit and a pumping apparatus for pumping fluid from the cooling unit through the inlet conduit and from the outlet conduit to the cooling unit.

In some embodiments, the pumping apparatus comprises (i) a first pump fluidly connected to the inlet conduit for pumping therethrough fluid from the cooling unit, (ii) a second pump fluidly connected to the outlet conduit for pumping therefrom fluid to the cooling unit, and (iii) a controller for operating the pumps at different rates to promote mixing through the fluid chamber. The outer wall of the catheter in the second region may comprise or consist essentially of a hard polymeric material.

The fluid chamber of the catheter may be coaxial with the urine-evacuation channel. For example, the fluid chamber may have an annular cross-section and is bounded by an outer surface of the urine-evacuation channel and an inner surface of the outer wall. Alternatively, the fluid chamber may have a pair of concentric channels separated by a porous membrane, where an outer channel of the fluid chamber is interfaced to the inlet conduit and an inner channel of the fluid chamber is interfaced to the outlet conduit. In other embodiments, the fluid chamber comprises a plurality of tortuously arranged channels.

In various embodiments, the fluid chamber of the catheter is coaxial with urine-evacuation channel. If desired, the catheter may include a temperature sensor and the system may include a controller, responsive to the temperature sensor, for controlling the pumping apparatus and the cooling unit.

Still another aspect of the invention pertains to a method for evacuating urine from a patient during a prostate procedure and cooling the prostate. In various embodiments, the method comprises providing a catheter comprising an elongated tube having an outer wall and a urine-evacuation lumen therethrough. The elongated tube includes a plurality of adjacent regions through which the urine-evacuation lumen passes, the regions including (i) a distal region including a terminal urine entry port in fluid communication with the urine-evacuation lumen and an inflatable balloon for retaining the port within the bladder, (ii) a cooling region proximally adjacent to the distal region and comprising a fluid chamber fluidically isolated from and surrounding the urine-evacuation lumen and in thermal contact with the outer wall of the catheter, and (iii) a proximal region proximally adjacent to the cooling region. The method further includes inserting the catheter through the patient's urinary tract until the distal portion of the catheter reaches the patient's bladder; inflating the balloon to retain the catheter in position, whereby the urine entry port is held within the patient's bladder and the cooling region spans the patient's prostate gland; draining urine from the patient's bladder via the urine-evacuation lumen; and circulating a cooling fluid through the fluid chamber of the catheter without disturbing a flow of urine through the urine-evacuation lumen.

In various embodiments, the fluid chamber of the catheter is coaxial with the urine-evacuation channel. For example, fluid chamber of the catheter may have an annular cross-section and be bounded by an outer surface of the urine-evacuation channel and an inner surface of the outer wall. Alternatively, the fluid chamber of the catheter may have a pair of concentric channels separated by a porous membrane, whereby an outer channel of the fluid chamber receives the cooling fluid and an inner channel ejects cooling fluid passing through the porous membrane. In other embodiments, the fluid chamber of the catheter comprises a plurality of tortuously arranged channels.

The method may further comprise the steps of sensing a temperature of the cooling fluid and, based at least in part thereon, controlling at least one of a cooling unit for the cooling fluid or the prostate procedure.

The term “substantially” or “approximately” means ±10% (e.g., by weight or by volume), and in some embodiments, ±5%. The term “consists essentially” of means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing will be more readily understood from the following detailed description of the invention, in particular, when taken in conjunction with the drawings, in which:

FIGS. 1A and 1B are sectional views showing the basic operation of a Foley catheter.

FIG. 2 is an elevation of a catheter in accordance with an embodiment of the present invention.

FIGS. 3A-3C axial sections of different segments of the catheter shown in FIG. 2.

FIGS. 4A-4C are transverse sections through alternative versions of the segment C-C′ shown in FIG. 3A, taken along the line D-D′.

### DETAILED DESCRIPTION

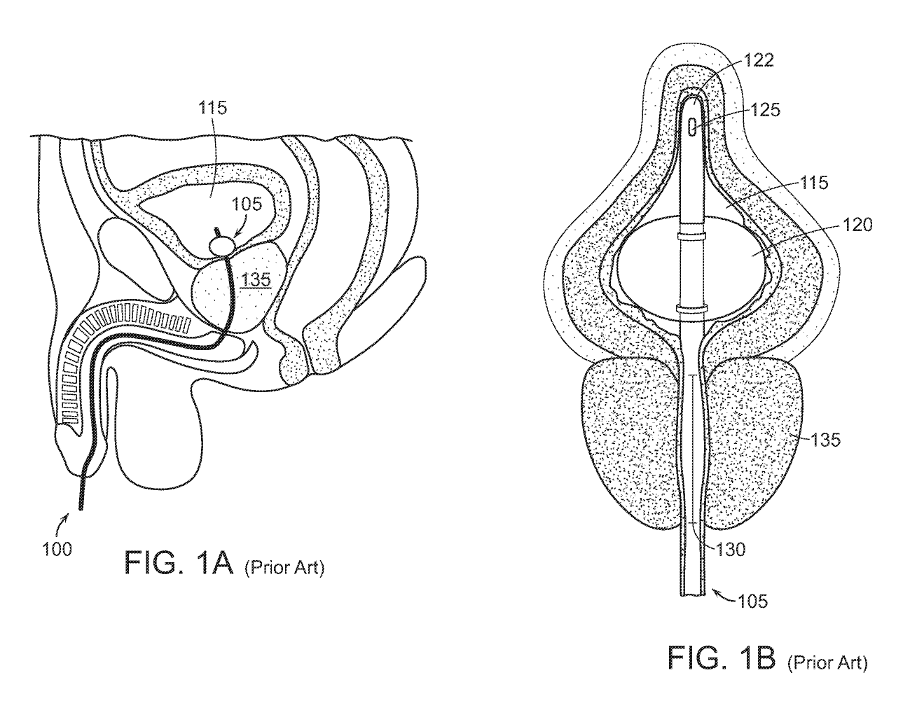

Refer first to FIGS. 1A and 1B, which illustrate the basic configuration and operation of a Foley catheter 100. The catheter passes through the patient's urinary tract until the distal portion 105 reaches the patient's bladder 115, at which point a balloon 120 is inflated (with air or water) to retain the distal end 122 within the bladder. Urine is evacuated through a collection port 110 and conducted through the lumen of the catheter 100, the proximal end of which is connected to a container (not shown) for collection. A segment 130 of the catheter 100 passes through the patient's prostate 135. Because this internal anatomy does not exhibit significant variation, the segment 130 is roughly the same length and in the same location across patients.

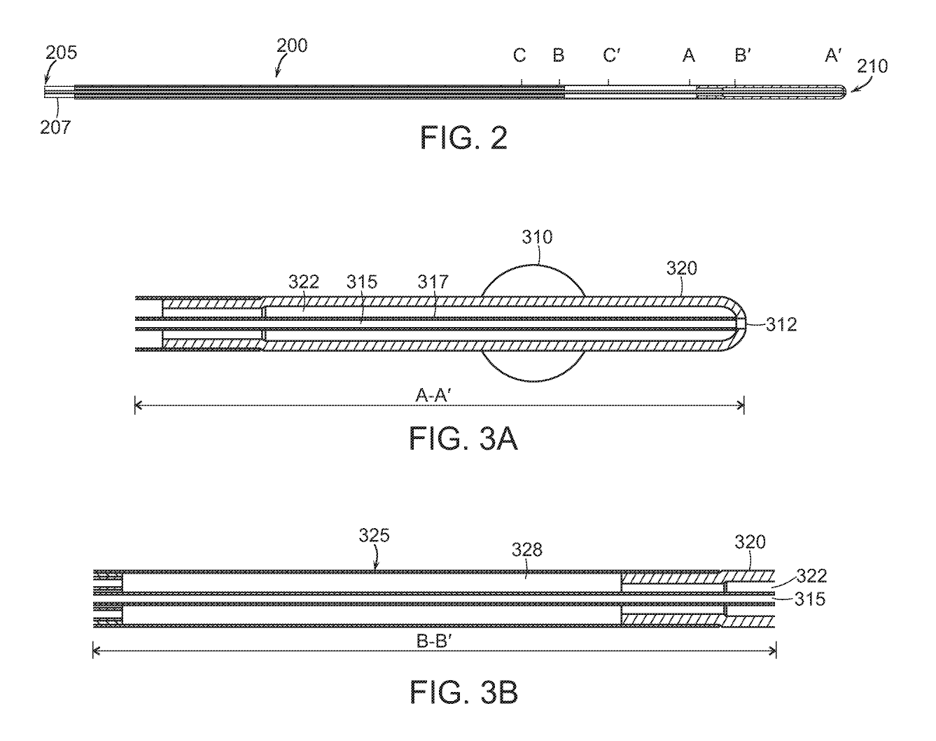

FIG. 2 illustrates a catheter 200 constructed in accordance with the principles of the present invention. Although the length is not shown to scale, the catheter 200 includes a proximal end 205 with a connector or interface 207 configured for attachment to a collection container and a distal end 210 with a collection port as described above. Points along the length of the catheter are labeled for reference in the ensuing discussion.

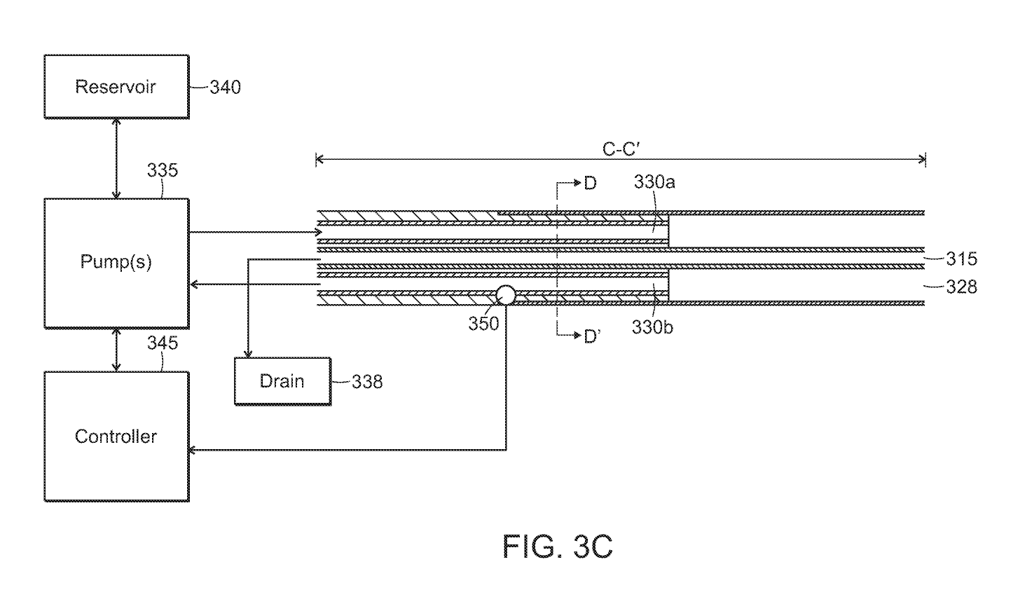

The segment A-A, shown sectionally in FIG. 3A, includes an inflatable balloon 310 and a collection port 312. A lumen 315 conducts urine through the catheter. Preferably, the lumen 315 is defined by a tube 317 made of an engineering plastic (e.g., KEVLAR, poly ether ketone (PEEK) and polysulfone) that serves both as a urine evacuation channel as well as conferring rigidity to the catheter. A high-strength plastic enables the diameter of the tube to be small without sacrificing rigidity. The diameter of the tube may be, for example, 1.25 mm with a lumen diameter of 0.75 mm. These dimensions are sufficiently small relative to the ultrasound wavelength that ultrasound waves will actually diffract around the tube 317). The tube 317 desirably exhibits high flexibility in transverse direction and high rigidity and strength in the longitudinal direction. This allows the catheter 200 to be used without a guide wire. The remainder of the catheter body, including the outer sheath 320, may be made from a flexible medical-grade polymer such as soft silicone. An interior tube 322, also made of soft silicone, may span the interior distance between the evacuation tube 317 and the exterior sheath 320. For ease of presentation, the conventional lumen used to inflate the balloon 310 is not shown or further discussed herein.

The interior tube 322 may terminate at or distal to the segment B-B′, which is illustrated in FIG. 3B and corresponds generally in terms of axial location to the prostate-spanning segment 130 shown in FIG. 1B, although it may be longer or shorter depending on the procedure with which the catheter 200 will be used. In the segment B-B′, an outer wall 325 surrounds and defines a hollow interior volume around and coaxial with the evacuation tube 317. This chamber 328 is filled, during operation, with a recirculating cooling fluid (typically chilled water) to cool the prostate tissue with which the segment B-B′ is in immediate contact.

The segment B-B′ should be substantially transparent to ultrasound waves and also provide good heat transfer. Thus, the outer wall 325 may be fabricated from very thin plastic with, optionally, a very thin (less than 0.3 mm) layer of silicone thereover. While thin, the outer wall 325 should be rigid in order to avoid radial expansion, which would stress and possibly injure the urinary tract. Suitable materials conferring the necessary rigidity and exhibiting substantial (e.g., >90%) transparency to ultrasound include polyethers such as MYLAR or a MYLAR composition or rigid (i.e., highly crosslinked and based on low-molecular-weight (<1000) polyols with more than two hydroxyl groups) polyurethane, which are strong, facilitating small wall thicknesses; wall thickness is minimized because both acoustic absorbence and heat transfer are inversely proportional thereto. The wall must nonetheless be sufficiently thick to resist deformation during use; typically the wall thickness is less than 5 mm and, in the case of MYLAR or rigid polyurethane, wall thicknesses of 2-3 mm are possible. Other suitable materials include polyethylene terephthalate (PET) and PET modifications as PET-C, polybutadiene terephthalate, etc. It should be stressed that ultrasound transparency and good heat transfer are particularly critical for segment B-B′, so in some embodiments, only this segment is made of a material exhibiting these properties, while the remainder of the catheter may be silicone or latex. In other embodiments, substantially the entire catheter is made of the same material.

Cooling fluid is delivered to and withdrawn from the chamber 328 by a pair of delivery conduits 330a, 330b in the segment C-C′ as shown in FIG. 3C, which are fluidically coupled at one end to the chamber 328 via the interface 207 (see FIG. 2) and at the other end to at least one pump 335. The tubes 330a, 330b are fluidically isolated from the evacuation tube 317, which empties into a drain or collection bag 338. The pump 335 is also fluidically connected to a chilling reservoir 340 for the recirculating cooling fluid, and its operation is governed by a controller 345. The controller 345 may optionally receive signals from, or interrogate, one or more sensors 350 within the catheter. In the illustrated embodiment, the sensor 350 is a temperature sensor (e.g., a thermocouple or thermistor) associated with the withdrawal tube 330b. By measuring the temperature of the cooling fluid after it has passed through the chamber, the controller 345 can adjust the rate of recirculation (e.g., increasing the rate to increase cooling efficiency) and/or the level of refrigeration in the reservoir 340 using conventional feedback programming. The sensor can also be used to verify proper operation of the system; for example, a sensed temperature above a threshold may indicate a component failure and signal danger to the patient, alerting the operator or shutting down the application of ultrasound.

The controller 345 may be provided as either software, hardware, or some combination thereof. For example, the system may be implemented on one or more conventional server-class computers, such as a PC having a CPU board containing one or more processors such as the Pentium or Celeron family of processors manufactured by Intel Corporation of Santa Clara, Calif., the 680×0 and POWER PC family of processors manufactured by Motorola Corporation of Schaumburg, Ill., and/or the ATHLON line of processors manufactured by Advanced Micro Devices, Inc., of Sunnyvale, Calif. The processor may also include a main memory unit for storing programs and/or data relating to the methods described above. The memory may include random access memory (RAM), read only memory (ROM), and/or FLASH memory residing on commonly available hardware such as one or more application specific integrated circuits (ASIC), field programmable gate arrays (FPGA), electrically erasable programmable read-only memories (EEPROM), programmable read-only memories (PROM), programmable logic devices (PLD), or read-only memory devices (ROM). In some embodiments, the programs may be provided using external RAM and/or ROM such as optical disks, magnetic disks, as well as other commonly used storage devices. For embodiments in which the functions are provided as one or more software programs, the programs may be written in any of a number of high level languages such as FORTRAN, PASCAL, JAVA, C, C++, C#, BASIC, various scripting languages, and/or HTML. Additionally, the software may be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM.

Depending on parameters such as the dimensions of the chamber 328, it may be desirable to encourage mixing and avoid the creation of a dead zone toward the distal end of the chamber—i.e., to create a mixing gradient whereby cooling diminishes with distance from the inlets of the delivery conduits 330a, 330b. For example, it is possible to cycle in-flow and out-flow to promote turbulence and hence mixing. For example, the pump 335 may actually be two pumps, each connected to one of the delivery conduits 330a, 330b and both connected to the reservoir 340. One pump drives cooling fluid into the chamber 328 while the other pumps fluid out of the chamber with the result of generating turbulence.

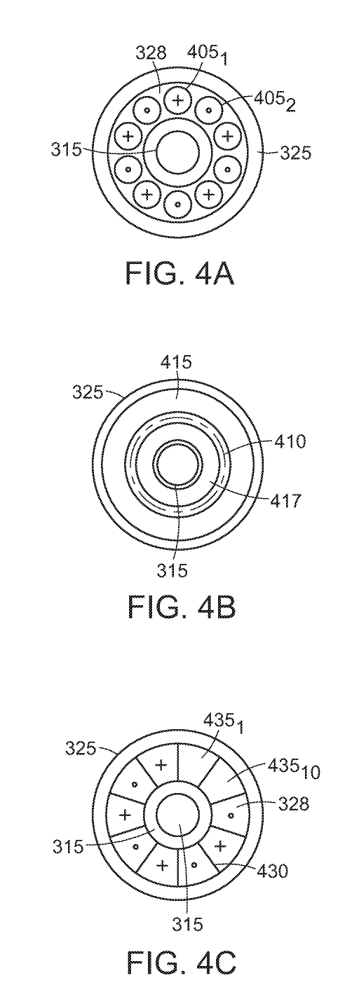

Cooling arrangements other than a unitary chamber are also possible. Transverse cross-sections of three representative alternatives are illustrated in FIGS. 4A-4C. In FIG. 4A, the open chamber volume is replaced with a plurality of tubes 405 distributed radially around the evacuation lumen 315. The flow directions of adjacent tubes are different, as indicated by the symbols (+) for the direction into the page and (−) for the direction out of the page. In this way, fresh cooling fluid is distributed radially and symmetrically around the catheter segment. Each of the tubes 405₁ (flow into the page) can be commonly connected to the inlet tube 330a (see FIG. 3C) and the tubes 405₂ (flow out of the page) can be commonly connected to the outlet tube 330b. In one implementation, each adjacent pair of tubes 405 is actually a single tube bent into a U shape at the distal end of the chamber 328 to define a two-level tortuous path therethrough.

Another arrangement, shown in FIG. 4B, utilizes a tubular porous barrier 410 concentric with the evacuation lumen 315, thereby forming an outer sleeve 415 into which cooling fluid is injected via the delivery conduit 330a and an inner sleeve 417 from which cooling fluid that has passed through the barrier 410 is withdrawn via the tube 330b. The pore size of the barrier 410 is selected to allow a desired circulation rate, and helps distribute fresh cooling fluid through the length of the sleeve 415.

With renewed reference to FIG. 4A, it is possible to utilize a single tube 405 that doubles back at each end of the chamber 328, defining a tortuous path through the chamber. The degree of cooling will diminish circumferentially from the inlet point, but depending on the circulation rate, this may not be problematic. A simpler tortuous-path alternative to this configuration is shown in FIG. 4C. Instead of a repeatedly bent internal tube, a series of longitudinal compartments is defined by a series of radial walls 430 symmetrically arranged around the urine-evacuation lumen 315 and extending from the outer wall of the lumen 315 to the inner surface of the wall 325. (It should be noted that the outer wall 325, as well as other walls described herein, can have multiple adjacent layers, which are within the scope of the term “wall” as used herein.) The walls 430 extend longitudinally from a barrier at one end of the chamber 328 but are spaced from the barrier at the opposite end in an alternating fashion, so that where a wall does not extend fully to one of the end barriers of the chamber 328, water will flow around it into the adjacent compartment. Thus, one wall 430 extends to the distal end of the chamber 328 but is spaced from the barrier at the proximal end, the next one is spaced from the barrier at the distal end but extends to the proximal end, and so on. A compartment 435₁ receives cooling fluid from the inlet tube 330a. The fluid circulates back and forth through the compartments until it passes through the last compartment 435₁₀, which is fluidically connected to the outlet tube 330b.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. In particular, embodiments of the invention need not include all of the features or have all of the advantages described herein. Rather, they may possess any subset or combination of features and advantages. Accordingly, the described embodiments are to be considered in all respects as only illustrative and not restrictive.

What is claimed is:

1. A catheter configured for evacuating urine from a patient during a prostate procedure, the catheter comprising an elongated tube for insertion through the patient's urinary tract, the tube having an outer wall and a urine-evacuation lumen therethrough and comprising a plurality of adjacent regions through which the urine-evacuation lumen passes, wherein:
a distal region is configured for insertion into the patient's bladder and includes a terminal urine entry port in fluid communication with the urine-evacuation lumen and an inflatable balloon for retaining the port within the bladder; and
at least a prostate-spanning region, proximally adjacent to the distal region and having a length sized to span the patient's prostate gland with the balloon inflated and the urine entry port within the patient's bladder, is substantially transparent to ultrasound.

2. The catheter of claim 1, further wherein:
a cooling region is coextensive with the prostate-spanning region and comprises a fluid chamber fluidically isolated from and surrounding the urine-evacuation lumen and in thermal contact with the outer wall of the catheter; and
a proximal region is proximally adjacent to the cooling region, the proximal region including (i) within the outer wall of the catheter, an inlet conduit in fluid communication with the fluid chamber for conducting a fluid thereto and and outlet conduit in fluid communication with the fluid chamber for conducting a fluid therefrom, and (ii) interfaces for fluidly coupling the urine-evacuation lumen to a collection container and the inlet and outlet conduits to a cooling and recirculation pump.

3. The catheter of claim 1, wherein the outer wall in the prostate-spanning region consists essentially of a hard polymeric material.

4. The catheter of claim 3, wherein the hard polymeric material is MYLAR.

5. The catheter of claim 3, wherein the hard polymeric material is a rigid polyurethane.

6. The catheter of claim 2, wherein the fluid chamber is coaxial with the urine-evacuation channel.

7. The catheter of claim 6, wherein the fluid chamber has an annular cross-section and is bounded by an outer surface of the urine-evacuation channel and an inner surface of the outer wall.

8. The catheter of claim 6, wherein the fluid chamber has a pair of concentric channels separated by a porous membrane, an outer channel of the fluid chamber being interfaced to the inlet conduit and an inner channel of the fluid chamber being interfaced to the outlet conduit.

9. The catheter of claim 2, wherein the fluid chamber comprises a plurality of tortuously arranged channels.

10. A system for evacuating urine from a patient during a prostate procedure and cooling the prostate, the system comprising:
a. a catheter comprising an elongated tube for insertion through the patient's urinary tract, the tube having an outer wall and a urine-evacuation lumen therethrough and comprising a plurality of adjacent regions through which the urine-evacuation lumen passes, wherein:
i. a distal region is configured for insertion into the patient's bladder and includes a terminal urine entry port in fluid communication with the urine-evacuation lumen and an inflatable balloon for retaining the port within the bladder;
ii. a cooling region is proximally adjacent to the distal region and has a length sized to span the patient's prostate gland with the balloon inflated and the urine entry port within the patient's bladder, the cooling region comprising a fluid chamber fluidically isolated from and surrounding the urine-evacuation lumen and in thermal contact with the outer wall of the catheter; and
iii. a proximal region is proximally adjacent to the cooling region, the proximal region including, within the outer wall of the catheter, an inlet conduit in fluid communication with the fluid chamber for conducting a fluid thereto and and outlet conduit in fluid communication with the fluid chamber for conducting a fluid therefrom;
b. a recirculation assembly configured to circulate a cooling fluid through the fluid chamber of the catheter without disturbing a flow of urine through the urine-evacuation lumen, the recirculation assembly comprising:
i. a cooling unit; and
ii. a pumping apparatus for pumping fluid from the cooling unit through the inlet conduit and from the outlet conduit to the cooling unit.

11. The system of claim 10, wherein the pumping apparatus comprises (i) a first pump fluidly connected to the inlet conduit for pumping therethrough fluid from the cooling unit, (ii) a second pump fluidly connected to the outlet conduit for pumping therefrom fluid to the cooling unit, and (iii) a controller for operating the pumps at different rates to promote mixing through the fluid chamber.

12. The system of claim 10, wherein the outer wall of the catheter in the second region consists essentially of a hard polymeric material.

13. The system of claim 10, wherein the fluid chamber of the catheter is coaxial with urine-evacuation channel.

14. The system of claim 13, wherein the fluid chamber of the catheter has an annular cross-section and is bounded by an outer surface of the urine-evacuation channel and an inner surface of the outer wall.

15. The system of claim 13, wherein the fluid chamber of the catheter has a pair of concentric channels separated by a porous membrane, an outer channel of the fluid chamber being interfaced to the inlet conduit and an inner channel of the fluid chamber being interfaced to the outlet conduit.

16. The system of claim 10, wherein the fluid chamber of the catheter comprises a plurality of tortuously arranged channels.

17. The system of claim 10, wherein the catheter further comprises a temperature sensor and the system further comprises a controller, responsive to the temperature sensor, for controlling the pumping apparatus and the cooling unit.

18. A method for evacuating urine from a patient during a prostate procedure and cooling the prostate, the method comprising the steps of:
a. providing a catheter comprising an elongated tube having an outer wall and a urine-evacuation lumen therethrough, the elongated tube comprising a plurality of adjacent regions through which the urine-evacuation lumen passes, the regions including (i) a distal region including a terminal urine entry port in fluid communication with the urine-evacuation lumen and an inflatable balloon for retaining the port within the bladder, (ii) a cooling region proximally adjacent to the distal region and comprising a fluid chamber fluidically isolated from and surrounding the urine-evacuation lumen and in thermal contact with the outer wall of the catheter, and (iii) a proximal region proximally adjacent to the cooling region;
b. inserting the catheter through the patient's urinary tract until the distal portion of the catheter reaches the patient's bladder;
c. inflating the balloon to retain the catheter in position, whereby the urine entry port is held within the patient's bladder and the cooling region spans the patient's prostate gland;
d. draining urine from the patient's bladder via the urine-evacuation lumen; and
e. circulating a cooling fluid through the fluid chamber of the catheter without disturbing a flow of urine through the urine-evacuation lumen.

19. The method of claim 18, wherein the fluid chamber of the catheter is coaxial with the urine-evacuation channel.

20. The method of claim 19, wherein the fluid chamber of the catheter has an annular cross-section and is bounded by an outer surface of the urine-evacuation channel and an inner surface of the outer wall.

21. The method of claim 19, wherein the fluid chamber of the catheter has a pair of concentric channels separated by a porous membrane, an outer channel of the fluid chamber receiving the cooling fluid and an inner channel ejecting cooling fluid passing through the porous membrane.

22. The method of claim 18, wherein the fluid chamber of the catheter comprises a plurality of tortuously arranged channels.

23. The method of claim 18, further comprising the steps of sensing a temperature of the cooling fluid and, based at least in part thereon, controlling at least one of a cooling unit for the cooling fluid or the prostate procedure.
